(19) United States
(12) Patent Application Publication
(10) Pub. No.: US 2019/0282063 A1
(43) Pub. Date: Sep. 19, 2019

(54) MEDICAL IMAGE PROCESSING APPARATUS, MEDICAL OBSERVATION APPARATUS, AND IMAGE PROCESSING METHOD

(71) Applicant: SONY OLYMPUS MEDICAL SOLUTIONS INC., Tokyo (JP)

(72) Inventor: Masataka KADO, Kanagawa (JP)

(73) Assignee: SONY OLYMPUS MEDICAL SOLUTIONS INC., Tokyo (JP)

(21) Appl. No.: 16/285,265

(22) Filed: Feb. 26, 2019

(30) Foreign Application Priority Data
Mar. 15, 2018 (JP) 2018-047492

(51) Int. Cl. A61B 1/00; A61B 90/00; A61B 34/30; A61B 1/045; G16H 30/40

(52) U.S. Cl. CPC A61B 1/00009; A61B 90/37; A61B 34/30; A61B 1/0676; A61B 1/045; G16H 30/40; A61B 1/00045

(57) ABSTRACT

There is provided a medical image processing apparatus including: an image processing section including a function of processing a medical captured image captured by an imaging device that images an observation target in correspondence with a target display apparatus. The image processing section rotates the medical captured image on the basis of a relative arrangement relationship of the target display apparatus with respect to a set primary display apparatus.

### CROSS REFERENCE TO RELATED APPLICATIONS

This application claims the benefit of Japanese Priority Patent Application JP 2018-047492 filed Mar. 15, 2018, the entire contents of which are incorporated herein by reference.

### BACKGROUND

The present disclosure relates to a medical image processing apparatus, a medical observation apparatus, and an image processing method.

Recently, in the medical field, to support microsurgery such as neurosurgical procedures, or to perform an endoscopic surgery, for example, medical observation apparatus capable of enlarged observation of an observation target such as an affected area are used in some cases. Examples of medical observation apparatus include a medical observation apparatus provided with an optical microscope, and a medical observation apparatus provided with an imaging device that functions as an electronic imaging microscope. In the following, the above medical observation apparatus provided with an optical microscope will be designated an “optical medical observation apparatus”. Also, in the following, the above medical observation apparatus provided with an imaging device will be designated an “electronic imaging medical observation apparatus” or simply a “medical observation apparatus” in some cases. Also, in the following, a captured image (a moving image or a still image; the same applies hereinafter) in which an observation target is captured by an imaging device provided in a medical observation apparatus is denoted a “medical captured image”.

With an electronic imaging medical observation apparatus, along with the increased image quality of imaging devices, the increased image quality of display apparatus on which captured images are displayed, and the like, the same or higher image quality than an optical medical observation apparatus has come to be obtained. Also, because a user who uses an electronic imaging medical observation apparatus (for example, medical personnel such as a surgeon or a surgeon's assistant) is not required to peer into an eyepiece lens included in an optical microscope like in the case of using an optical medical observation apparatus, it is possible to move the position of the imaging device more freely. For this reason, using an electronic imaging medical observation apparatus has an advantage of enabling more flexible support of microsurgery, and in the medical field, utilization of electronic imaging medical observation apparatus is progressing.

In such circumstances, technologies related to display apparatus used in endoscopic surgery are being developed. One example of a “technology for causing the display screen of a display apparatus to rotate clockwise and counter-clockwise on the basis of a rotation request from a practitioner using a screen rotation operating unit” is the technology described in JP 2008-517703T.

### SUMMARY

In surgery in which a medical observation apparatus is used, multiple surgeons carry out procedures by looking at a medical captured image captured by a single imaging device in some cases. To give an example in which procedures are carried out by two surgeons, such as an operating surgeon and an assistant for example, a display apparatus is disposed in front of each of the operating surgeon and the assistant. At this time, the vertical direction of the imaging device typically is matched to the direction of the operating surgeon.

Here, at the position of the operating surgeon, the top and bottom of the imaging device, the direction in which the surgeon is facing, and the direction in which the display apparatus is disposed are aligned. For this reason, the operating surgeon's own hands appear at the bottom of the display screen of the display apparatus disposed in front of the operating surgeon, and the operating surgeon is able to carry out procedures without a sense of discomfort.

However, at the position of the assistant, the top and bottom of the imaging device, the direction in which the surgeon is facing, and the direction in which the display apparatus is disposed are not aligned. For this reason, the assistant's own hands do not appear at the bottom of the display screen of the display apparatus disposed in front of the assistant, the vertical direction inside the display screen is misaligned with the direction in which the surgeon is facing, and as a result, intuitive procedures by the assistant may be impeded in some cases.

As illustrated by the above example, in surgery in which a medical observation apparatus is used, there is a possibility that some medical personnel members, such as an assistant, may be unable to perform intuitive procedures. Additionally, being unable to perform intuitive procedures using the medical observation apparatus may lead to reduced convenience for the medical personnel members using the medical observation apparatus, for example.

The present disclosure proposes a novel and improved medical image processing apparatus, medical observation apparatus, and image processing method capable of achieving an improvement in convenience for medical personnel members.

According to an embodiment of the present disclosure, there is provided a medical image processing apparatus including: an image processing section including a function of processing a medical captured image captured by an imaging device that images an observation target in correspondence with a target display apparatus. The image processing section rotates the medical captured image on the basis of a relative arrangement relationship of the target display apparatus with respect to a set primary display apparatus.

In addition, according to an embodiment of the present disclosure, there is provided a medical observation apparatus including: an imaging device configured to image an observation target; and an image processing section including a function of processing a medical captured image captured by the imaging device in correspondence with a target display apparatus. The image processing section rotates the medical captured image on the basis of a relative arrangement relationship of the target display apparatus with respect to a set primary display apparatus.

In addition, according to an embodiment of the present disclosure, there is provided an image processing method, executed by a medical image processing apparatus, including: processing a medical captured image captured by an imaging device that images an observation target in correspondence with a target display apparatus. The processing rotates the medical captured image on the basis of a relative arrangement relationship of the target display apparatus with respect to a set primary display apparatus.

According to an embodiment of the present disclosure, an improvement in convenience for medical personnel members may be achieved.

Note that the effects described above are not necessarily limitative. With or in the place of the above effects, there may be achieved any one of the effects described in this specification or other effects that may be grasped from this specification.

### BRIEF DESCRIPTION OF THE DRAWINGS

FIG. 1 is an explanatory diagram illustrating a first example of a configuration of a medical observation system according to the present embodiment;

FIG. 2 is an explanatory diagram illustrating a first example of a display apparatus included in the medical observation system according to the present embodiment;

FIG. 3 is an explanatory diagram illustrating one example of the arrangement of the display apparatus illustrated in FIG. 2;

FIG. 4 is an explanatory diagram illustrating a second example of a display apparatus included in the medical observation system according to the present embodiment;

FIG. 5 is an explanatory diagram illustrating a third example of a display apparatus included in the medical observation system according to the present embodiment;

FIG. 6 is an explanatory diagram for explaining an example of the configuration of an imaging device provided in a medical observation apparatus according to the present embodiment;

FIG. 7 is an explanatory diagram illustrating a second example of a configuration of a medical observation system according to the present embodiment;

FIG. 8 is a function block diagram illustrating one example of a configuration of a medical observation apparatus according to the present embodiment;

FIG. 9 is an explanatory diagram for explaining an overview of the image processing method according to the present embodiment;

FIG. 10 is an explanatory diagram for explaining an overview of the image processing method according to the present embodiment; and

FIG. 11 is an explanatory diagram for explaining one example of processes related to the image processing method according to the present embodiment.

### DETAILED DESCRIPTION OF THE EMBODIMENT(S)

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

The description hereinafter will proceed in the following order.

1. Medical observation system according to present embodiment and image processing method according to present embodiment
[1] Configuration of medical observation system
   [1-1] Medical observation system according to first example
   [1-2] Medical observation system according to second example
[2] Image processing method according to present embodiment
   [2-1] Overview of image processing method according to present embodiment
   [2-2] Processes related to image processing method according to present embodiment
[3] Example of advantageous effects exhibited by use of image processing method according to present embodiment

2. Program according to present embodiment

### Medical Observation System According to Present Embodiment and Image Processing Method According to Present Embodiment

Hereinafter, an example of a medical observation system according to the present embodiment will be described, while an image processing method according to the present embodiment will also be described.

Hereinafter, the case in which the medical observation apparatus according to the present embodiment executes processes related to the image processing method according to the present embodiment, that is, the case in which the medical observation apparatus according to the present embodiment functions as a medical image processing apparatus will be described primarily. Note that in the medical observation system according to the present embodiment, the apparatus that functions as the medical image processing apparatus is not limited to the medical observation apparatus according to the present embodiment. For example, in the medical observation system according to the present embodiment, the display apparatus described later may also function as the medical image processing apparatus that executes the processes related to the image processing method according to the present embodiment. For example, in the medical observation system according to the present embodiment, any apparatus capable of executing the processes related to the image processing method according to the present embodiment, such as a medical controller, may function as the medical image processing apparatus.

### [1] Configuration of Medical Observation System

### [1-1] Medical Observation System According to First Example

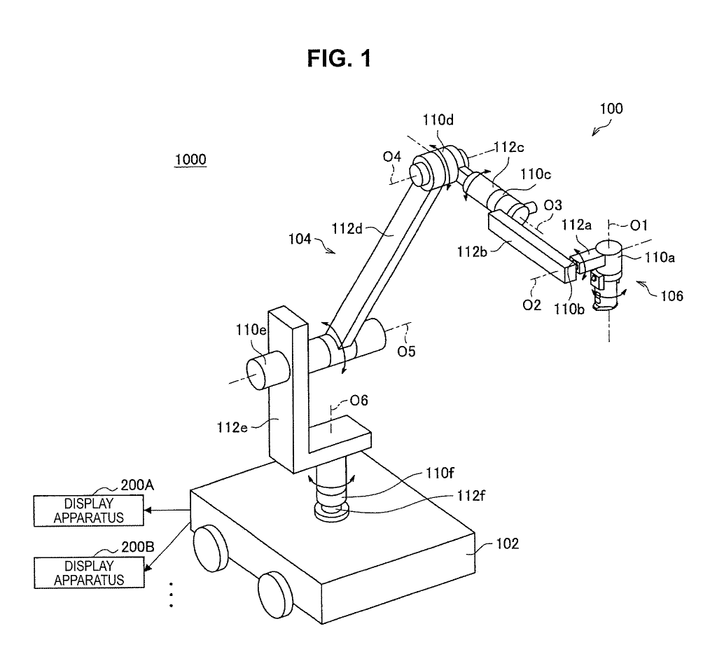

FIG. 1 is an explanatory diagram illustrating a first example of the configuration of a medical observation system 1000 according to the present embodiment. The medical observation system 1000 illustrated in FIG. 1 includes the medical observation apparatus 100 and multiple display apparatus 200A, 200B, and so on, for example. In the following, the “display apparatus 200” will be used as a collective term for the multiple display apparatus 200A, 200B, and so on, or to denote a single display apparatus from among the multiple display apparatus 200A, 200B, and so on in some cases.

Note that the medical observation system according to the first example is not limited to the example illustrated in FIG. 1.

For example, the medical observation system according to the first example additionally may include a medical control apparatus (not illustrated) that controls various operations in the medical observation apparatus 100. In the medical observation system 1000 illustrated in FIG. 1, as described later, an example is illustrated in which, by providing the medical observation apparatus 100 with a control section (described later), the medical observation apparatus 100 includes the functions of the medical control apparatus (not illustrated).

Examples of the medical control apparatus (not illustrated) include, a “medical controller”, a “computer such as a server”, and the like. Also, the medical control apparatus (not illustrated) may be, for example, an integrated circuit (IC) that can be embedded in equipment like the above.

Also, the medical observation system according to the first example may also have a configuration including multiple medical observation apparatus 100. In the case in which the medical observation system according to the first example includes multiple medical observation apparatus 100, the processes related to the image processing method described later are executed in each medical observation apparatus 100. Also, in the case in which the medical observation system according to the first example has a configuration including multiple medical observation apparatus 100, each medical observation apparatus 100 may be associated with multiple display apparatus 200. In the case in which multiple medical observation apparatus 100 are associated with a single display apparatus 200, in the display apparatus 200, by performing a switching operation or the like for switching the medical captured image to be displayed on the display screen, for example, it is possible to switch which medical captured image captured in which medical observation apparatus 100 is to be displayed on the display screen.

Hereinafter, each apparatus included in the medical observation system 1000 according to the first example illustrated in FIG. 1 will be described.

### [1-1-1] Display Apparatus 200

The display apparatus 200 is a display device in the medical observation system 1000 according to the first example, and corresponds to an external display device from the perspective of the medical observation apparatus 100. The display apparatus 200 displays various images on a display screen, such as a medical captured image taken in the medical observation apparatus 100, or an image related to a user interface (UI), for example. Also, the display apparatus 200 may include a configuration capable of 3D display according to any method. The display on the display apparatus 200 is controlled by, for example, the medical observation apparatus 100 or the medical control apparatus (not illustrated).

In the medical observation system 1000, the display apparatus 200 is installed in an arbitrary location visible to a person involved in a surgery inside an operating room, such as on a wall, the ceiling, or the floor of the operating room.

Examples of the display apparatus 200 include a liquid crystal display, an organic electro-luminescence (EL) display, a cathode ray tube (CRT) display, and the like.

Note that the display apparatus 200 is not limited to the example illustrated above. For example, the display apparatus 200 may also be an arbitrary wearable apparatus that is used by being worn on the body of the surgeon or the like, such as a head-mounted display, an eyewear-type apparatus, or the like.

The display apparatus 200 runs on electric power supplied from an internal power source such as a battery provided in the display apparatus 200, on electric power supplied from a connected external power source, or the like, for example.

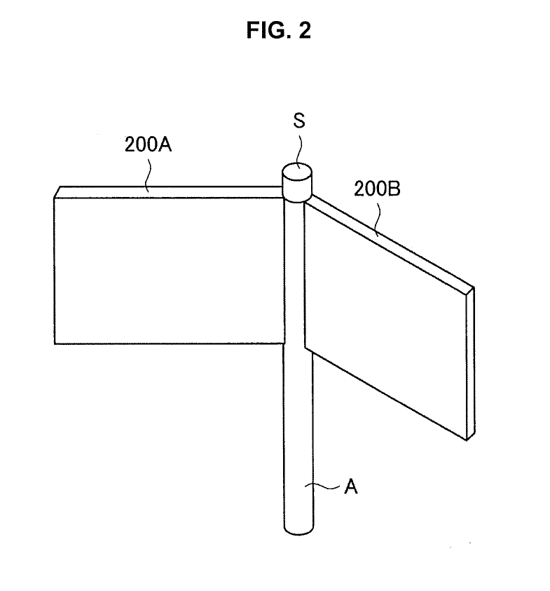

FIG. 2 is an explanatory diagram illustrating a first example of the display apparatus 200 included in the medical observation system 1000 according to the present embodiment, and illustrates one example of a configuration in which two display apparatus 200A and 200B are supported by an arm A. In the example illustrated in FIG. 2, a single angle sensor S is provided. The angle sensor S detects the angle obtained between the display apparatus 200A and the display apparatus 200B on the arm A.

The angle sensor S may be a rotary encoder, for example. The rotary encoder may be an absolute rotary encoder or an incremental rotary encoder. Also, the angle sensor S may be any type of sensor capable of obtaining a rotational angle, such as a rotational angular velocity sensor. The angle detected by the angle sensor S is the relative angle between the display apparatus 200A and the display apparatus 200B. Like the example illustrated in FIG. 2, if it is possible to specify the positions where the display apparatus 200A and the display apparatus 200B are provided, the relative arrangement relationship between the display apparatus 200A and the display apparatus 200B is specified (or the relative arrangement relationship is estimated) by the relative angle between the display apparatus 200A and the display apparatus 200B.

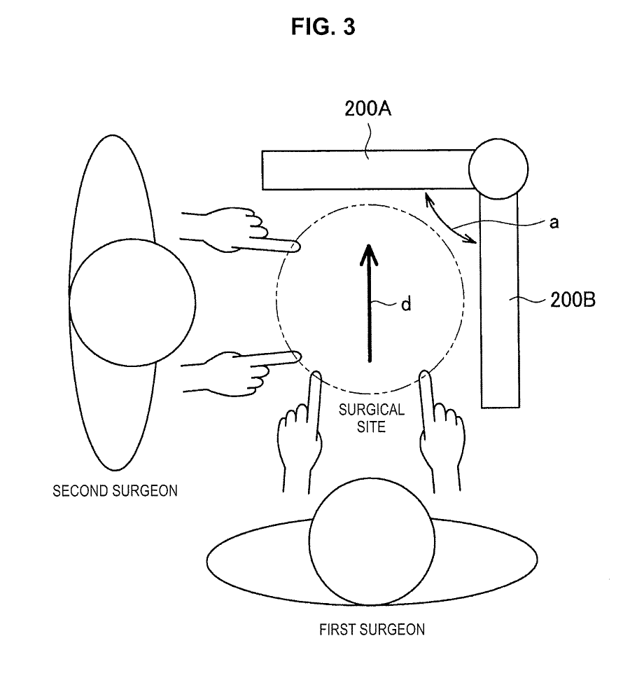

FIG. 3 is an explanatory diagram illustrating one example of the arrangement of the display apparatus 200 illustrated in FIG. 2. As illustrated in FIG. 3, the display apparatus 200A is disposed in front of a first surgeon, who is one surgeon who carries out procedures, and the display apparatus 200 is disposed in front of a second surgeon, who is another surgeon who carries out procedures. For example, in the case in which the first surgeon is the operating surgeon, as indicated by the sign d in FIG. 3, the vertical direction of the imaging device is matched to the direction of the first surgeon. Also, the relative arrangement relationship between the display apparatus 200A and the display apparatus 200B may be expressed by the angle labeled with the sign a in FIG. 3, and this angle is detected by the angle sensor S. Note that depending on the positional relationship of the surgeons, the display apparatus 200B may be disposed in front of the first surgeon and the display apparatus 200A may be disposed in front of the second surgeon.

The angle a is detected with reference to the first arrangement relationship below, for example.
First arrangement relationship: an arrangement relationship in which the display screen of the display apparatus 200A and the display screen of the display apparatus 200B are facing in opposite directions (for example, an arrangement relationship in which the display screen of the display apparatus 200A and the display screen of the display apparatus 200B are facing each other)

In the case in which the relative arrangement relationship between the display apparatus 200A and the display apparatus 200B is the above first arrangement relationship, the angle a is a reference angle=0[°]. The range that the angle a may take is from 0[°] to ±180[°]. Herein, for example, “+” indicates a counter-clockwise (left-hand; the same applies hereinafter) rotational angle, while “−” indicates a clockwise (right-hand; the same applies hereinafter) rotational angle. In other words, the angle a may take a range from 0[°] to 360[°].

Note that the relative arrangement relationship between the display apparatus 200A and the display apparatus 200B is not limited to being detected with reference to the above first arrangement relationship.

For example, the relative arrangement relationship between the display apparatus 200A and the display apparatus 200B may also be detected with reference to the second arrangement relationship below, for example.
Second arrangement relationship: an arrangement relationship in which the display screen of the display apparatus 200A and the display screen of the display apparatus 200B are facing in the same direction

In the case in which the relative arrangement relationship between the display apparatus 200A and the display apparatus 200B is detected with reference to the above second arrangement relationship, the relative arrangement relationship between the display apparatus 200A and the display apparatus 200B is expressed by an angle b (not illustrated) that becomes a reference angle=0[°] in the case of a=180[°] for the angle a illustrated in FIG. 3.

The angle b is detected by the angle sensor S. The range that the angle b may take is from 0[°] to ±180[°]. Herein, for example, “+” indicates a counter-clockwise rotational angle, while “−” indicates a clockwise rotational angle. In other words, the angle b may take a range from 0[°] to 360[°].

Note that the relative arrangement relationship between the display apparatus 200A and the display apparatus 200B is not limited to being expressed by an angle with reference to the above first arrangement relationship or the above second arrangement relationship. For example, the relative arrangement relationship between the display apparatus 200A and the display apparatus 200B may also be detected by treating the angle when the display apparatus 200A and the display apparatus 200B are in any chosen arrangement relationship as a reference angle=0[°].

The following mainly takes as an example the case in which the relative arrangement relationship between the display apparatus 200A and the display apparatus 200B is expressed by the angle a illustrated in FIG. 3.

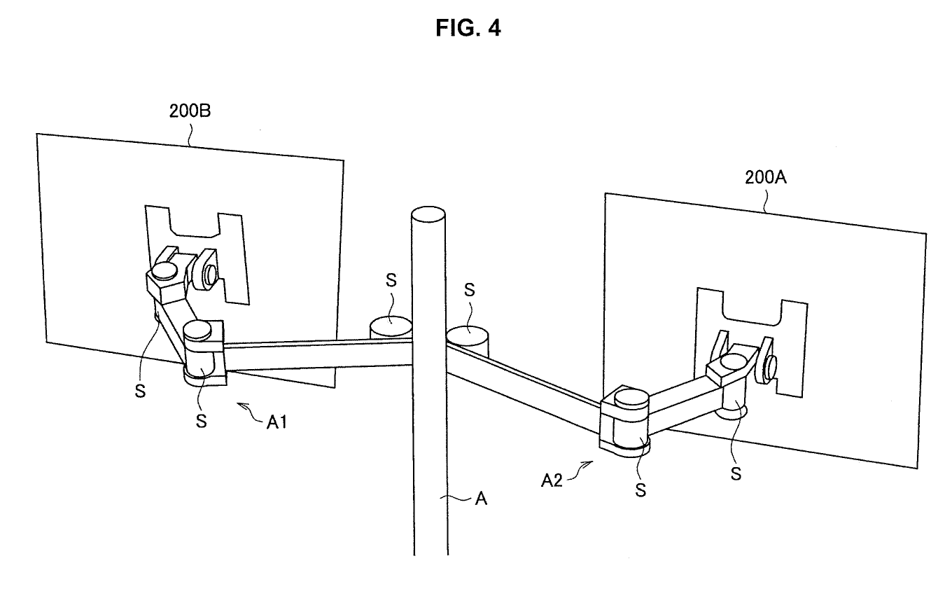

FIG. 4 is an explanatory diagram illustrating a second example of the display apparatus 200 included in the medical observation system 1000 according to the present embodiment, and illustrates another example of a configuration in which two display apparatus 200A and 200B are supported by the arm A. The example illustrated in FIG. 4 illustrates a “configuration in which arms A1 and A2 having three joints are rotatably connected to the arm A, and each of the display apparatus 200A and 200B is supported by the arm A1 or the arm A2”. Also, in the example illustrated in FIG. 4, the angle sensor S is provided for each of the six joints.

Even with a configuration in which the angle sensor S is provided for each joint as illustrated in FIG. 4 for example, by combining the detection results of the multiple angle sensors S, it is possible to specify the relative angle between the display apparatus 200A and the display apparatus 200B. Therefore, even in the example illustrated in FIG. 4, it is possible to specify the relative arrangement relationship between the display apparatus 200A and the display apparatus 200B. The above process of specifying the relative angle between the display apparatus 200 on the basis of the combination of detection results from the multiple angle sensors S may be executed by the medical observation apparatus 100 or by an apparatus external to the medical observation apparatus 100, such as a medical control apparatus (not illustrated).

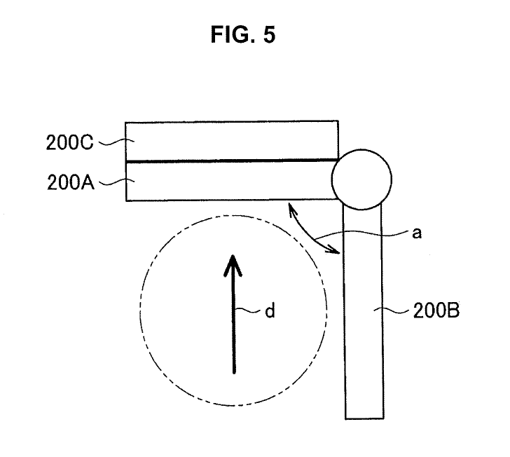

FIG. 5 is an explanatory diagram illustrating a third example of the display apparatus 200 included in the medical observation system 1000 according to the present embodiment, and illustrates one example of a configuration in which three display apparatus 200A, 200B, and 200C are provided. In FIG. 5, in addition to the configuration illustrated in FIG. 2, a display apparatus 200C is provided on the back (the opposite side from the display screen) of the display apparatus 200A. Also, as indicated by the sign d in FIG. 5, in the example illustrated in FIG. 5, similarly to the example illustrated in FIG. 3, the vertical direction of the imaging device is matched to the direction of the first surgeon (not illustrated in FIG. 5).

In the example illustrated in FIG. 5, similarly to the example illustrated in FIG. 2, a single angle sensor S detects the angle obtained between the display apparatus 200A and the display apparatus 200B on the arm A. Also, in the example illustrated in FIG. 5, the angle obtained between the display apparatus 200A and the display apparatus 200C is set to 180[°]. By setting the angle obtained between the display apparatus 200A and the display apparatus 200C, it is possible to use the detection result from the angle sensor S to specify the angle obtained between the display apparatus 200B and the display apparatus 200C. Therefore, in the example illustrated in FIG. 5, it is likewise possible to specify each of the relative arrangement relationship between the display apparatus 200A and the display apparatus 200B, the relative arrangement relationship between the display apparatus 200A and the display apparatus 200C, and the relative arrangement relationship between the display apparatus 200B and the display apparatus 200C.

The display apparatus 200 included in the medical observation system 1000 have the configuration described with reference to FIGS. 2 to 5, for example.

Note that the number of display apparatus 200 included in the medical observation system 1000 obviously is not limited to the examples described with reference to FIGS. 2 to 5. Also, the arrangement relationship of the display apparatus 200 included in the medical observation system 1000 is not limited to the examples described with reference to FIGS. 2 to 5. For example, the display apparatus 200 may also be arranged vertically at positions of different heights from the floor of the medical setting.

Also, the number of angle sensors S used to specify the relative arrangement relationship between two display apparatus 200 is not limited to the example illustrated in FIG. 2, the example illustrated in FIG. 4, and the example illustrated in FIG. 5, and may be any number of sensors enabling the specification of a relative angle between the display apparatus 200 to specify the relative arrangement relationship.

Also, in the examples illustrated with reference to FIGS. 2 to 5, examples in which the relative arrangement relationship of the display apparatus 200 is specified on the basis of an angle between the display apparatus 200 detected by the angle sensor S such as a rotary encoder is illustrated, but the method of specifying the relative arrangement relationship of the display apparatus 200 is not limited to the examples illustrated above.

For example, in the medical observation system 1000, by analyzing a captured image captured by an imaging device such as a surgical field camera, the relative arrangement relationship of the display apparatus 200 inside the space may be specified. In other words, the angle sensor according to the present embodiment may also be an imaging device. The relative arrangement relationship of the display apparatus 200 inside the space is specified by, for example, detecting the display apparatus 200 from a captured image by utilizing any type of object detection technology capable of detecting the display apparatus 200 from an image, and estimating the angle between the detected display apparatus 200. The above process of specifying the relative arrangement relationship of the display apparatus 200 inside the space on the basis of a captured image may be executed by the medical observation apparatus 100 or by an apparatus external to the medical observation apparatus 100, such as a medical control apparatus (not illustrated).

Also, in the medical observation system 1000, by analyzing a distance image obtained by a distance sensor, for example, the relative arrangement relationship of the display apparatus 200 inside the space may be specified. In other words, the angle sensor according to the present embodiment may also be a distance sensor. The above process of specifying the relative arrangement relationship of the display apparatus 200 inside the space on the basis of a distance image may be executed by the medical observation apparatus 100 or by an apparatus external to the medical observation apparatus 100, such as a medical control apparatus (not illustrated).

Also, in the examples illustrated in FIGS. 3 and 5, as indicated by the sign d in FIGS. 3 and 5, an example in which the vertical direction of the imaging device is matched to the direction of the first surgeon is illustrated, but the vertical direction of the imaging device may also be matched to the second surgeon. One example of a case of matching the vertical direction of the imaging device to the direction of the second surgeon is the case in which the second surgeon is the operating surgeon.

### [1-1-2] Medical Observation Apparatus 100

The medical observation apparatus 100 illustrated in FIG. 1 is an electronic imaging medical observation apparatus. For example, in the case in which the medical observation apparatus 100 illustrated in FIG. 1 is used during surgery, the surgeon (one example of the user of the medical observation apparatus 100) observes an operating site (an affected area) while referring to a medical captured image which has been taken by the medical observation apparatus 100 and displayed on the display screen of the display apparatus 200, and performs various treatments, such as techniques depending on the surgical procedure, on the operating site.

As illustrated in FIG. 1, the medical observation apparatus 100 is provided with a base 102, an arm 104, and an imaging device 106, for example.

Additionally, although not illustrated in FIG. 1, the medical observation apparatus 100 may also be provided with, for example, one or multiple processors (not illustrated) including a computational circuit such as a microprocessing unit (MPU), read-only memory (ROM; not illustrated), random access memory (RAM; not illustrated), a recording medium (not illustrated), and a communication device (not illustrated). The medical observation apparatus 100 runs on electric power supplied from an internal power source such as a battery provided in the medical observation apparatus 100, on electric power supplied from a connected external power source, or the like, for example.

The one or multiple processors (not illustrated) function as the control section in the medical observation apparatus 100 (described later). The ROM (not illustrated) stores programs and control data such as computational parameters used by the one or multiple processors (not illustrated). The RAM (not illustrated) temporarily stores programs executed by the one or multiple processors (not illustrated), or the like.

The recording medium (not illustrated) functions as a storage section (not illustrated) in the medical observation apparatus 100. A variety of data is stored on the recording medium (not illustrated), including data related to the image processing method according to the present embodiment, and various applications, for example. Herein, examples of the recording medium (not illustrated) include a magnetic recording medium such as a hard disk, non-volatile memory such as flash memory, and the like. Additionally, the recording medium (not illustrated) may also be removable from the medical observation apparatus 100.

The communication device (not illustrated) is a communication device provided in the medical observation apparatus 100, and fulfills a role of communicating in a wireless or wired manner with an external apparatus such as the display apparatus 200. Herein, examples of the communication device (not illustrated) include an IEEE 802.15.1 port and transmitting-receiving circuit (wireless communication), an IEEE 802.11 port and transmitting-receiving circuit (wireless communication), a communication antenna and a RF circuit (wireless communication), a LAN terminal and a transmitting-receiving circuit (wired communication), and the like.

### [1-1-2-1] Base 102

The base 102 is the base of the medical observation apparatus 100. One end of the arm 104 is connected to the base 102, and the base 102 supports the arm 104 and the imaging device 106.

Also, casters are provided on the base 102, for example, and the medical observation apparatus 100 contacts the floor through the casters. By providing the casters, the medical observation apparatus 100 is able to move easily over the floor by the casters.

### [1-1-2-2] Arm 104

The arm 104 includes multiple links joined to each other by joint sections.

In addition, the arm 104 supports the imaging device 106. The imaging device 106 supported by the arm 104 is movable three-dimensionally, and after moving, the position and the attitude of the imaging device 106 are maintained by the arm 104.

More specifically, the arm 104 includes, for example, multiple joint sections 110a, 110b, 110c, 110d, 110e, and 110f, and multiple links 112a, 112b, 112c, 112d, 112e, and 112f rotatably joined to each other by the joint sections 110a, 110b, 110c, 110d, 110e, and 110f. The rotatable range of each of the joint sections 110a, 110b, 110c, 110d, 110e, and 110f is set arbitrarily during the design stage, the manufacturing stage, or the like so that the desired motion of the arm 104 is realized.

In other words, in the medical observation apparatus 100 illustrated in FIG. 1, six degrees of freedom are realized in relation to the movement of the imaging device 106 by six rotation axes (first axis O1, second axis O2, third axis O3, fourth axis O4, fifth axis O5, and sixth axis O6) corresponding to the six joint sections 110a, 110b, 110c, 110d, 110e, and 110f included in the arm 104. More specifically, in the medical observation apparatus 100 illustrated in FIG. 1, motion with six degrees of freedom, including three degrees of translational freedom and three degrees of rotational freedom, is realized.

Actuators (not illustrated) are provided in each of the joint sections 110a, 110b, 110c, 110d, 110e, and 110f. Each of the joint sections 110a, 110b, 110c, 110d, 110e, and 110f rotates about the corresponding rotation axis by the driving of the actuators (not illustrated). The driving of the actuators (not illustrated) is controlled by, for example, a processor that functions as the control section described later, or an external medical control apparatus (not illustrated).

Each of the joint sections 110a, 110b, 110c, 110d, 110e, and 110f may be provided with angle sensors (not illustrated) capable of detecting a rotational angle for each of six rotation axes. The angle sensors may be, for example, rotary encoders, or any sensors capable of obtaining a rotational angle for each of six rotation axes, such as angular velocity sensors.

By having each of the joint sections 110a, 110b, 110c, 110d, 110e, 110f rotate about the corresponding rotation axis by the driving of the actuators (not illustrated), various operations of the arm 104, such as extending and contracting (folding up) the arm 104, for example, are realized.

The joint section 110a has an approximately cylindrical shape, and supports the imaging device 106 (the top end of the imaging device 106 in FIG. 1) on the front end portion of the joint section 110a (the bottom end portion in FIG. 1), so as to allow revolution about a rotation axis (first axis O1) parallel to the central axis of the imaging device 106. Herein, the medical observation apparatus 100 is configured so that the first axis O1 is aligned with the optical axis in the imaging device 106. In other words, by causing the imaging device 106 to revolve about the first axis O1 illustrated in FIG. 1, the medical captured image captured by the imaging device 106 becomes an image which has changed so that the field of view rotates.

The link 112a is an approximately rod-shaped member, and securely supports the joint section 110a. The link 112a extends in a direction orthogonal to the first axis O1, for example, and is connected to the joint section 110b.

The joint section 110b has an approximately cylindrical shape, and supports the link 112a so as to allow revolution about a rotation axis (second axis O2) orthogonal to the first axis O1. Also, the link 112b is securely connected to the joint section 110b.

The link 112b is an approximately rod-shaped member, and extends in a direction orthogonal to the second axis O2. Also, each of the joint section 110b and the joint section 110c is connected to the link 112b.

The joint section 110c has an approximately cylindrical shape, and supports the link 112b so as to allow revolution about a rotation axis (third axis O3) mutually orthogonal to each of the first axis O1 and the second axis O2. Also, one end of the link 112c is securely connected to the joint section 110c.

Herein, by having the front end side (the side on which the imaging device 106 is provided) of the arm 104 revolve about the second axis O2 and the third axis O3, the imaging device 106 can be made to move so that the position of the imaging device 106 in the horizontal plane is changed. In other words, in the medical observation apparatus 100, controlling the rotation about the second axis O2 and the third axis O3 makes it possible to move the field of view of the medical captured image in a flat plane.

The link 112c is a member in which one end has an approximately cylindrical shape, and the other end has an approximately rod-like shape. On the side of the one end of the link 112c, the joint section 110c is securely connected so that the central axis of the joint section 110c and the central axis of the approximately cylindrical shape are the same. Also, on the side of the other end of the link 112c, the joint section 110d is connected.

The joint section 110d has an approximately cylindrical shape, and supports the link 112c so as to allow revolution about a rotation axis (fourth axis O4) orthogonal to the third axis O3. The link 112d is securely connected to the joint section 110d.

The link 112d is an approximately rod-shaped member, and extends orthogonally to the fourth axis O4. One end of the link 112d is securely connected to the joint section 110d so as to abut the approximately cylindrical side face of the joint section 110d. Also, the joint section 110e is connected to the other end of the link 112d (the end on the opposite side of the side where the joint section 110d is connected).

The joint section 110e has an approximately cylindrical shape, and supports one end of the link 112d so as to allow revolution about a rotation axis (fifth axis O5) parallel to the fourth axis O4. Also, one end of the link 112e is securely connected to the joint section 110e.

Herein, the fourth axis O4 and the fifth axis O5 are rotation axis about which the imaging device 106 may be moved in the vertical direction. By having the front end side (the side on which the imaging device 106 is provided) of the arm 104 revolve about the fourth axis O4 and the fifth axis O5, the position of the imaging device 106 in the vertical direction changes. Thus, by having the front end side (the side on which the imaging device 106 is provided) of the arm 104 revolve about the fourth axis O4 and the fifth axis O5, changing the distance between the imaging device 106 and an observation target, such as an operating site of a patient, becomes possible.

The link 112e is a member that includes a combination of a first member having an approximate L-shape with one edge extending in the vertical direction while the other edge extends in the horizontal direction, and a rod-like second member that extends vertically downward from the part of the first member that extends in the horizontal direction. The joint section 110e is securely connected to the part of the first member of the link 112e that extends in the vertical direction. Also, the joint section 110f is connected to the second member of the link 112e.

The joint section 110f has an approximately cylindrical shape, and supports the link 112e so as to allow revolution about a rotation axis (sixth axis O6) parallel to the vertical direction. Also, the link 112f is securely connected to the joint section 110f.

The link 112f is an approximately rod-shaped member, and extends in the vertical direction. The joint section 110f is connected to one end of the link 112f. Also, the other end of the link 112f (the end on the opposite side of the side where the joint section 110f is connected) is securely connected to the base 102.

By having the arm 104 include the configuration indicated above, in the medical observation apparatus 100, six degrees of freedom are realized with respect to the movement of the imaging device 106.

Note that the configuration of the arm 104 is not limited to the example indicated above.

For example, each of the joint sections 110a, 110b, 110c, 110d, 110e, and 110f of the arm 104 may be provided with a brake that restrains rotation in each of the joint sections 110a, 110b, 110c, 110d, 110e, and 110f. The brake according to the present embodiment may be a brake of an arbitrary method, such as a mechanically driven brake or an electrically driven electromagnetic brake, for example.

The driving of the above brakes is controlled by, for example, a processor that functions as the control section described later, or an external medical control apparatus (not illustrated). By controlling the driving of the above brakes, in the medical observation apparatus 100, the operating mode of the arm 104 is set. Examples of operating modes of the arm 104 include a locked mode and a free mode.

Herein, the locked mode according to the present embodiment is, for example, an operating mode in which the position and the attitude of the imaging device 106 are locked by using brakes to restrain rotation about each rotation axis provided in the arm 104. By having the arm 104 enter the locked mode, the operating state of the medical observation apparatus 100 becomes a locked state in which the position and the attitude of the imaging device 106 are locked.

Also, the free mode according to the present embodiment is an operating mode in which the above brakes are released, thereby allowing each rotation axis provided in the arm 104 to rotate freely. For example, in the free mode, the position and the attitude of the imaging device 106 are adjustable by direct operations performed by the surgeon. Herein, a direct operation according to the present embodiment means, for example, an operation in which the surgeon grips the imaging device 106 with his or her hand, and directly moves the imaging device 106.

### [1-1-2-3] Imaging Device 106

The imaging device 106 is supported by the arm 104, and images an observation target such as an operating site of a patient, for example. Imaging in the imaging device 106 is controlled by, for example, a processor that functions as the control section described later, or an external medical control apparatus (not illustrated).

The imaging device 106 has a configuration corresponding to an electronic imaging microscope, for example.

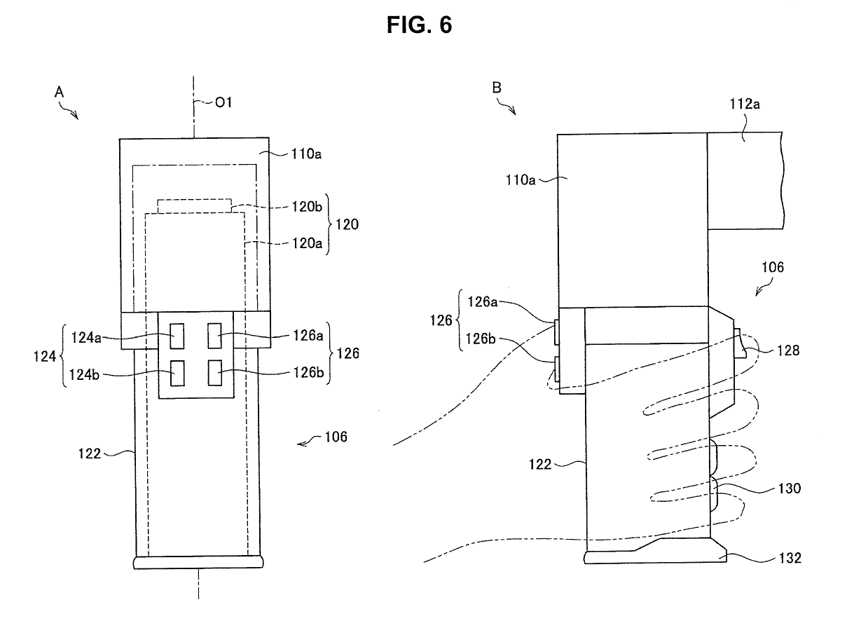

FIG. 6 is an explanatory diagram for explaining an example of the configuration of the imaging device 106 provided in the medical observation apparatus 100 according to the present embodiment.

For example, the imaging device 106 includes an imaging member 120 and a barrel member 122 having an approximately cylindrical shape, with the imaging member 120 being provided inside the barrel member 122.

On an aperture on the bottom end of the barrel member 122 (the lower end in FIG. 6), for example, a cover glass (not illustrated) for protecting the imaging member 120 is provided.

Additionally, for example, a light source (not illustrated) is provided inside the barrel member 122, and during imaging, the subject is irradiated with illuminating light radiating from the light source through the cover glass. Reflected light (observation light) from the subject irradiated with illuminating light enters the imaging member 120 through the cover glass (not illustrated), whereby an image signal indicating the subject (an image signal indicating a medical captured image) is obtained by the imaging member 120.

As the imaging member 120, any of various known types of configurations used in an electronic imaging microscope section can be applied.

To give one example, the imaging member 120 includes an optical system 120a and an image sensor 120b including an imaging element that takes an image of an observation target with light transmitted through the optical system 120a, for example. The optical system 120a includes optical elements such as a mirror and one or multiple lenses, such as an objective lens, a zoom lens, and a focus lens, for example. Examples of the image sensor 120b include an image sensor using multiple imaging elements, such as a complementary metal-oxide semiconductor (CMOS) or a charge-coupled device (CCD).

The imaging member 120, by including two or more imaging devices provided with an optical system 120a and an image sensor 120b, functions as what is called a stereo camera.

Each imaging device included in the imaging member 120 is equipped with one or multiple functions typically provided in an electronic imaging microscope section, such as a zoom function (one or both of an optical zoom function and an electronic zoom function) and an autofocus (AF) function.

In addition, the imaging member 120 may also be configured to be capable of imaging at what are called high resolutions, such as 4K and 8K, for example. By configuring the imaging member 120 to be capable of imaging at high resolutions, it becomes possible to ensure a predetermined resolution (such as full HD image quality, for example), while also displaying an image on the display apparatus 200 having a large display screen, such as 50 inches or more, for example. For this reason, visibility is improved for the surgeon watching the display screen. Also, by configuring the imaging member 120 to be capable of imaging at high resolutions, even if the captured image is enlarged by the electronic zoom function and displayed on the display screen of the display apparatus 200, it is still possible to ensure a predetermined resolution. Furthermore, in the case of using the electronic zoom function to ensure a predetermined resolution, since it is possible to reduce the performance of the optical zoom function in the imaging device 106, the optical system of the imaging device 106 can be simplified, and the imaging device 106 can be configured more compactly.

In the imaging device 106, for example, various operating devices for controlling the operation of the imaging device 106 are provided. For example, in FIG. 6, a zoom switch 124, a focus switch 126, and an operating mode change switch 128 are provided on the imaging device 106. Note that the positions and shapes in which to provide the zoom switch 124, the focus switch 126, and the operating mode change switch 128 obviously are not limited to the example illustrated in FIG. 6.

The zoom switch 124 and the focus switch 126 are an example of an operating device for adjusting the imaging parameters in the imaging device 106.

The zoom switch 124 includes, for example, a zoom-in switch 124a that increases the zoom magnification (enlargement ratio), and a zoom-out switch 124b that decreases the zoom magnification. By performing an operation on the zoom switch 124, the zoom magnification is adjusted, and the zoom is adjusted.

The focus switch 126 includes, for example, a long-range focus switch 126a that increases the focal length to the observation target (subject), and a close-range focus switch 126b that decreases the focal length to the observation target. By performing an operation on the focus switch 126, the focal length is adjusted, and the focus is adjusted.

The operating mode change switch 128 is an example of an operating device for changing the operating mode of the arm 104 in the imaging device 106. By performing an operation on the operating mode change switch 128, the operating mode of the arm 104 is changed. Examples of operating modes of the arm 104 include a locked mode and a free mode, as described above.

One example of an operation with respect to the operating mode change switch 128 is an operation of pressing the operating mode change switch 128. For example, the operating mode of the arm 104 becomes the free mode while the surgeon is pressing the operating mode change switch 128, and the operating mode of the arm 104 becomes the locked mode when the surgeon is not pressing the operating mode change switch 128.

In addition, the imaging device 106 is provided with, for example, an anti-slip member 130 and a projecting member 132 in order to further raise operability, convenience, and the like when an operator who performs operations on various operation devices performs an operation.

The anti-slip member 130 is a member provided to prevent slipping of an operating body such as a hand when, for example, the operator performs an operation on the barrel member 122 with the operating body. The anti-slip member 130 is formed with a material having a large coefficient of friction, for example, and has a slip-resistant structure due to unevenness or the like.

The projecting member 132 is member provided to prevent an operating body such as a hand blocking the field of view of the optical system 120a when the operator performs an operation on the barrel member 122 with the operating body, or to prevent a cover glass (not illustrated) from becoming dirty due to the cover glass being contacted by the operating body when an operation is performed with the operating body.

Note that the position and shape in which each of the anti-slip member 130 and the projecting member 132 is provided obviously are not limited to the example illustrated in FIG. 6. In addition, the imaging device 106 does not have to be provided with one or both of the anti-slip member 130 and the projecting member 132.

The image signal (image data) generated by imaging in the imaging device 106 is subjected to image processing in a processor that functions as the control section described later, for example. Examples of image processing according to the present embodiment include one or multiple processes from among various processes such as gamma correction, white balance adjustment, image enlargement or reduction related to the electronic zoom function, and pixel interpolation, for example. Also, the image processing according to the present embodiment may include the processes related to the image processing method described later, for example.

Note that in the case in which the medical observation system according to the present embodiment includes a medical control apparatus (not illustrated) that controls various operations in the medical observation apparatus 100, the image processing according to the present embodiment may also be performed in the medical control apparatus (not illustrated). In this case, the medical control apparatus (not illustrated) functions as a medical image processing apparatus capable of executing the processes related to the image processing method according to the present embodiment.

For example, the medical observation apparatus 100 transmits a display control signal and the image signal subjected to image processing as described above to the display apparatus 200.

By transmitting the display control signal and the image signal to the display apparatus 200, on the display screen of the display apparatus 200, a medical captured image in which the observation target is imaged (for example, a captured image in which the operating site is imaged) is displayed enlarged or reduced at a desired magnification by one or both of the optical zoom function and the electronic zoom function.

The medical observation apparatus 100 illustrated in FIG. 1 includes the hardware configuration illustrated with reference to FIGS. 1 and 6, for example.

Note that the hardware configuration of the medical observation apparatus according to the present embodiment is not limited to the configuration illustrated with reference to FIGS. 1 and 6.

For example, the medical observation apparatus according to the present embodiment may also be a configuration not provided with the base 102, in which the arm 104 is directly attached to the ceiling, a wall, or the like of the operating room or the like. For example, in the case in which the arm 104 is attached to the ceiling, the medical observation apparatus according to the present embodiment becomes a configuration in which the arm 104 hangs down from the ceiling.

Also, although FIG. 1 illustrates an example in which the arm 104 is configured so that six degrees of freedom are realized with respect to the driving of the imaging device 106, the configuration of the arm 104 is not limited to a configuration whereby the degrees of freedom with respect to the driving of the imaging device 106 become six degrees of freedom. For example, it is sufficient to configure the arm 104 so that the imaging device 106 can move appropriately in accordance with the application, and factors such as the number and arrangement of joint sections and links, and the directions of the drive shafts of the joint sections can be set appropriately so that the arm 104 has the desired degrees of freedom.

Also, although FIGS. 1 and 6 illustrate an example in which various types of operating devices for controlling the operation of the imaging device 106 are provided on the imaging device 106, some or all of the operating devices illustrated in FIGS. 1 and 6 may also not be provided on the imaging device 106. To give one example, the various types of operating devices for controlling the operation of the imaging device 106 may also be provided in another part other than the imaging device 106 included in the medical observation apparatus according to the present embodiment. Also, to give another example, the various types of operating devices for controlling the operation of the imaging device 106 may also be external operating devices, such as a footswitch or a remote controller.

Additionally, the imaging device 106 may also have a configuration enabling switching among multiple observation modes. Observation modes according to the present embodiment may include, for example, an observation mode that executes imaging with natural light, an observation mode that executes imaging with special light, an observation mode that executes imaging by utilizing an image-enhancing observation technology such as narrow-band imaging (NBI), and the like. Special light according to the present embodiment refers to light in a specific wavelength band, such as light in the fluorescent wavelength band of fluorescent observation using 5-Aminolevulinic acid (5-ALA).

One example of the configuration of the imaging device 106 enabling switching among multiple observation modes is a “configuration provided with a filter that allows light of a specific wavelength band to pass through while not allowing light of other wavelength bands to pass through, and a movement mechanism that selectively disposes the filter on the optical path”, for example. The specific wavelength band that the filter according to the present embodiment allows to pass through may be, for example, the wavelength band of near-infrared rays (for example, the wavelength band from approximately 0.7 [micrometers] to 2.5 [micrometers]), the fluorescent wavelength band for fluorescent observation using 5-ALA (for example, the wavelength band from approximately 0.6 [micrometers] to 0.65 [micrometers]), the fluorescent wavelength band of indocyanine green (ICG) (for example, the wavelength band from approximately 0.82 [micrometers] to 0.85 [micrometers]), or the like.

Note that the imaging device 106 may also be provided with multiple filters that allow different wavelength bands to pass through. Also, although the above illustrates an example in which imaging is executed with the light of a specific wavelength band by disposing a filter on the optical path, the configuration of the imaging device 106 for executing imaging with the light of a specific wavelength band obviously is not limited to the example illustrated above.

### [1-2] Medical Observation System According to Second Example

The medical observation system 1000 according to the present embodiment is not limited to the configuration illustrated in the first example illustrated in FIG. 1. Next, as another example of the medical observation system 1000, one example of a configuration of the medical observation system 1000 including the medical observation apparatus 100 that functions as an endoscopic apparatus will be described.

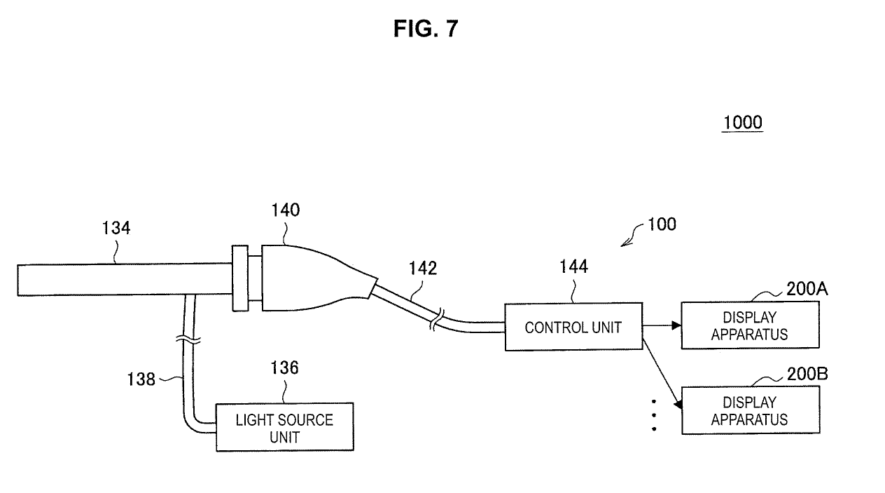

FIG. 7 is an explanatory diagram illustrating a second example of the configuration of the medical observation system 1000 according to the present embodiment. The medical observation system 1000 illustrated in FIG. 7 includes the medical observation apparatus 100 and multiple display apparatus 200A, 200B, and so on, for example. In the case in which the medical observation apparatus 100 illustrated in FIG. 7 is used during surgery, the surgeon observes the surgical site while referring to a medical captured image captured by the medical observation apparatus 100 and displayed on the display screen of the display apparatus 200, and performs various treatments, such as techniques depending on the surgical procedure, on the surgical site.

Note that the medical observation system according to the second example is not limited to the example illustrated in FIG. 7.

For example, the medical observation system according to the second example additionally may include a medical control apparatus (not illustrated) that controls various operations in the medical observation apparatus 100, similarly to the medical observation system according to the first example.

Also, the medical observation system according to the second example may be a configuration including a plurality of the medical observation apparatus 100, similarly to the medical observation system according to the first example.

Hereinafter, each apparatus included in the medical observation system 1000 according to the second example illustrated in FIG. 7 will be described.

### [1-2-1] Display Apparatus 200

The display apparatus 200 is a display device in the medical observation system 1000 according to the second example, and corresponds to an external display device from the perspective of the medical observation apparatus 100. The display apparatus 200 included in the medical observation system 1000 according to the second example is similar to the display apparatus 200 included in the medical observation system 1000 according to the first example.

### [1-2-2] Medical Observation Apparatus 100

The medical observation apparatus 100 illustrated in FIG. 7 is provided with an insertion member 134, a light source unit 136, a light guide 138, a camera head 140, a cable 142, and a control unit 144, for example. The medical observation apparatus 100 runs on electric power supplied from an internal power source such as a battery provided in the medical observation apparatus 100, on electric power supplied from a connected external power source, or the like, for example.

The insertion member 134 has an elongated shape, and is internally provided with an optical system that condenses incident light. The front end of the insertion member 134 is inserted inside a body cavity of a patient. The rear end of the insertion member 134 is detachably connected to the front end of the camera head 140. Also, the insertion member 134 is connected to the light source unit 136 through the light guide 138, and is supplied with light from the light source unit 136.

The insertion member 134 may be formed with an inflexible material or a flexible material. Depending on the material used to form the insertion member 134, the medical observation apparatus 100 may be called a rigid scope or a flexible scope.

The light source unit 136 is connected to the insertion member 134 through the light guide 138. The light source unit 136 supplies light to the insertion member 134 through the light guide 138.

For example, the light source unit 136 includes multiple light sources that emit light of different wavelengths. The multiple light sources included in the light source unit 136 may be, for example, a light source that emits red light, a light source that emits green light, and a light source that emits blue light. The light source that emits red light may be one or multiple red light-emitting diodes, for example. The light source that emits green light may be one or multiple green light-emitting diodes, for example. The light source that emits blue light may be one or multiple blue light-emitting diodes, for example. Note that the multiple light sources included in the light source unit 136 obviously are not limited to the example illustrated above. For example, the light source unit 136 includes the multiple light sources on a single chip or includes the multiple light sources on multiple chips.

The light source unit 136 is connected to the control unit 144 in a wired or wireless manner, and the light emission in the light source unit 136 is controlled by the control unit 144.

Light supplied to the insertion member 134 is emitted from the front end of the insertion member 134, and irradiates an observation target such as tissue inside the body cavity of the patient. Additionally, reflected light from the observation target is condensed by the optical system inside the insertion member 134.

The camera head 140 has a function of imaging the observation target. The camera head 140 is connected to the control unit 144 through a signal transmission member, namely the cable 142.

The camera head 140 includes an image sensor, images the observation target by photoelectrically converting the reflected light from the observation target condensed by the insertion member 134, and outputs an image signal obtained by the imaging (a signal expressing the medical captured image) to the control unit 144 through the cable 142. The image sensor included in the camera head 140 may be, for example, an image sensor using multiple imaging elements such as CMOS and CCD elements.

In the medical observation apparatus 100 that functions as an endoscopic apparatus, for example, the insertion member 134, the light source unit 136, and the camera head 140 fulfill the role of an “imaging device that is inserted inside a body of a patient and images the inside of the body”.

Note that the medical observation apparatus 100 that functions as an endoscopic apparatus may also be a configuration provided with multiple imaging devices that function as what is called a stereo camera.

The control unit 144 controls the imaging device. More specifically, the control unit 144 controls each of the light source unit 136 and the camera head 140.

Also, the control unit 144 includes a communication device (not illustrated), and transmits an image signal output from the camera head 140 to the display apparatus 200 by any form of wireless communication or any form of wired communication. The control unit 144 may also transmit an image signal and a display control signal to the display apparatus 200.

The communication device (not illustrated) included in the control unit 144 may be, for example, an IEEE 802.15.1 port and a transmitting-receiving circuit (wireless communication), an IEEE 802.11 port and a transmitting-receiving circuit (wireless communication), a communication antenna and an RF circuit (wireless communication), an optical communication device (wireless communication or wired communication), a LAN terminal and a transmitting-receiving circuit (wired communication), or the like. The communication device (not illustrated) may also be a configuration capable of communicating with one or multiple external apparatus by multiple communication methods.

In addition, the control unit 144 may execute predetermined processing on the image signal output from the camera head 140, and transmit the image signal that has been subjected to the predetermined processing to the display apparatus 200. The predetermined processing on the image signal may be, for example, white balance adjustment, image enlargement or reduction according to an electronic zoom function, pixel interpolation, and the like. Additionally, the predetermined processing on the image signal may also include the processes related to the image processing method described later, for example.

Note that the control unit 144 may also store a medical captured image based on the image signal.

The control unit 144 may be a camera control unit (CCU), for example.

The medical observation apparatus 100 that functions as an endoscopic apparatus includes the hardware configuration illustrated with reference to FIG. 7, for example. In the medical observation apparatus 100 that functions as an endoscopic apparatus, for example, the insertion member 134, the light source unit 136, and the camera head 140 fulfill the role of the imaging device, and imaging in the imaging device is controlled by the control unit 144.

### [1-3] Functional Configuration of Medical Observation Apparatus 100

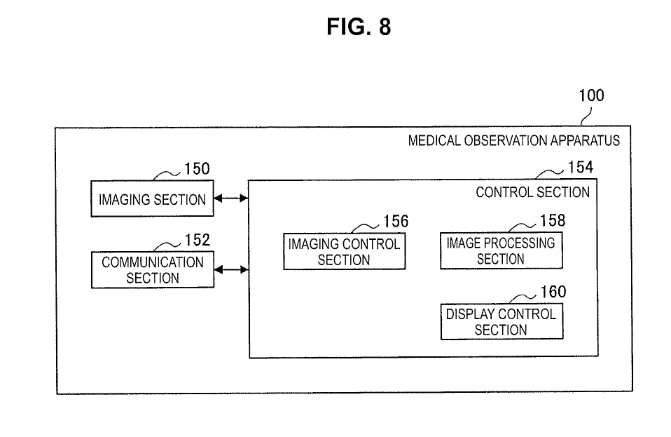

Next, the medical observation apparatus 100 illustrated in FIGS. 1 and 7 will be described using function blocks. FIG. 8 is a function block diagram illustrating one example of the configuration of the medical observation apparatus 100 according to the present embodiment.

For example, the medical observation apparatus 100 is provided with an imaging section 150, a communication section 152, and a control section 154.

The imaging section 150 images the observation target. For example, the imaging section 150 includes the “imaging device 106” (in the case of the medical observation apparatus 100 illustrated in FIG. 1), or the “insertion member 134, the light source unit 136, and the camera head 140” (in the case of the medical observation apparatus 100 illustrated in FIG. 7). Imaging in the imaging section 150 is controlled by the control section 154, for example.

The communication section 152 is a communication device provided in the medical observation apparatus 100, and fulfills a role of communicating in a wireless or wired manner with an external apparatus such as the display apparatus 200. The communication section 152 includes the communication device (not illustrated) described above, for example. Communication in the communication section 152 is controlled by the control section 154, for example.

The control section 154 includes the processor (not illustrated) described above, for example, and fulfills a role of controlling the medical observation apparatus 100 overall. In addition, the control section 154 fulfills a role of leading the execution of the processes related to the image processing method described later. Note that the processes related to the image processing method in the control section 154 may also be executed in a distributed manner by multiple processing circuits (such as multiple processors, for example).

More specifically, the control section 154 includes an imaging control section 156, an image processing section 158, and a display control section 160, for example.

The imaging control section 156 controls the imaging device included in the imaging section 150. Examples of the control of the imaging device include control of one or multiple functions typically provided in an electronic imaging microscope section, such as control of an AF function, including at least a zoom function (one or both of an optical zoom function and an electronic zoom function).

The image processing section 158 executes the processes related to the image processing method described later, and processes the medical captured image in correspondence with the display apparatus 200 targeted by the processes related to the image processing method (hereinafter designated the “target display apparatus”). The target display apparatus may be a preset fixed display apparatus 200 or may be set in any way by an operation by a user who uses the medical observation apparatus 100. One example of the processes related to the image processing method according to the present embodiment will be described later.

For example, the display control section 160 controls the display on the display apparatus 200 by conveying the display control signal and the image signal to the communication device (not illustrated) included in the communication section 152, and causing the display control signal and the image signal to be transmitted to the display apparatus 200. The image signal that the display control section 160 causes to be transmitted may include an image signal after the processes related to the image processing method are executed in the image processing section 158. Note that the control of communication in the communication section 152 may also be performed by a communication control section (not illustrated) included in the control section 154.

For example, by including the image processing section 158, the control section 154 fulfills a role of leading the execution of the processes related to the image processing method according to the present embodiment. Also, for example, by including the imaging control section 156 and the display control section 160, the control section 154 fulfills a role of controlling the medical observation apparatus 100 overall.

Note that the functional configuration of the control section 154 is not limited to the example illustrated in FIG. 8.

For example, it is possible for the control section 154 to have any configuration corresponding to how the functions included in the medical observation apparatus 100 are divided up, such as a configuration corresponding to how the processes related to the image processing method according to the present embodiment are divided up.

To give one example, in the case in which the medical observation apparatus 100 has the configuration illustrated in FIG. 1, the control section 154 additionally may include an arm control section (not illustrated) that controls the driving of the arm 104. One example of control of the driving of the arm 104 includes, for example, “applying a control signal that controls driving to the actuators (not illustrated) corresponding to each of the joint sections 110a, 110b, 110c, 110d, 110e, and 110f”, and the like.

The medical observation apparatus 100 performs processes related to the image processing method according to the present embodiment described later with the functional configuration illustrated in FIG. 8, for example.

Note that the functional configuration of the medical observation apparatus according to the present embodiment is not limited to the configuration illustrated in FIG. 8.

For example, in the medical observation apparatus according to the present embodiment, some or all of the imaging control section 156, the image processing section 158, and the display control section 160 illustrated in FIG. 8 can be provided separately from the control section 154 (for example, realized by a different processing circuit).

Additionally, in the medical observation apparatus according to the present embodiment, the functional configuration capable of executing the processes related to the image processing method according to the present embodiment is not limited to the configuration illustrated in FIG. 8, and it is possible for the medical observation apparatus according to the present embodiment to take a functional configuration corresponding to how the processes related to the image processing method according to the present embodiment are divided up.

Also, in the case in which the medical observation apparatus according to the present embodiment has the configuration illustrated in FIG. 1, the medical observation apparatus according to the present embodiment includes an arm section (not illustrated) including the arm 104. The arm 104 included in the arm section (not illustrated) supports the imaging device 106 included in the imaging section 150.

Also, for example, in the case of communicating with an external apparatus via an external communication device having a function and configuration similar to the communication section 152, the medical observation apparatus according to the present embodiment may also not be provided with the communication section 152.

Also, in the case in which the medical observation system according to the present embodiment includes a medical control apparatus (not illustrated), and the medical observation apparatus according to the present embodiment is controlled by the medical control apparatus (not illustrated), the medical observation apparatus according to the present embodiment may also not be provided with the control section 154.

Herein, the medical control apparatus (not illustrated) is, for example, provided with a control section having a function and configuration similar to the control section 154, and thereby executes processes related to the image processing method according to the present embodiment described later, and in addition, controls the operation in each structural element such as the imaging section 150 provided in the medical observation apparatus according to the present embodiment. The medical control apparatus (not illustrated) communicates with the medical observation apparatus according to the present embodiment via a provided communication device or a connected external communication device, and thereby controls the operation in each structural element provided in the medical observation apparatus according to the present embodiment.

Furthermore, in the case in which the medical observation system according to the present embodiment includes the medical control apparatus (not illustrated), and the medical observation apparatus according to the present embodiment is controlled by the medical control apparatus (not illustrated), it is also possible for the medical observation apparatus according to the present embodiment to take a configuration that does not include some of the functions of the control section 154.

### [2] Image Processing Method According to Present Embodiment

Next, the image processing method according to the present embodiment will be described. The following gives an example of a case in which the processes related to the image processing method according to the present embodiment are executed by the medical observation apparatus 100 (more specifically, the control section 154 included in the medical observation apparatus 100, for example). Note that, as described above, in the medical observation system according to the present embodiment, the processes related to the image processing method according to the present embodiment may also be executed by the display apparatus 200, a medical control apparatus (not illustrated), or the like.

### [2-1] Summary of Image Processing Method According to Present Embodiment

As described above, in surgery in which a medical observation apparatus is used, a medical captured image captured by a single imaging device is displayed on the display screen of a display apparatus corresponding to each of multiple surgeons, and each surgeon carries out procedures by looking at the corresponding display screen in some cases. However, in the above case, for a subset of the surgeons, the vertical direction inside the display screen is misaligned with the direction in which the surgeon is facing, and as a result, intuitive procedures may be impeded. Also, being unable to perform intuitive procedures using the medical observation apparatus may lead to reduced convenience for the medical personnel members using the medical observation apparatus, for example.

Accordingly, the medical observation apparatus 100 processes the medical captured image in correspondence with the target display apparatus and rotates the medical captured image corresponding to the target display apparatus. The medical captured image corresponding to the target display apparatus is the medical captured image displayed on the display screen of the target display apparatus (or the medical captured image to be displayed on the display screen of the target display apparatus). Control of the display of the medical captured image on the display screen of the target display apparatus may be executed by the medical observation apparatus 100 or may be executed by the display apparatus 200, a medical control apparatus (not illustrated), or the like.

By rotating the medical captured image corresponding to the target display apparatus, it becomes possible to match the vertical direction inside the display screen to the direction in which a surgeon is facing, and the “vertical direction inside the display screen being misaligned with the direction in which the surgeon is facing” as above may be prevented.

Therefore, by using the image processing method according to the present embodiment, an improvement in convenience for the medical personnel members may be achieved. Also, in the case in which the image processing method according to the present embodiment is used, since the impeding of intuitive procedures by the medical personnel members is prevented, it is possible to contribute to an improvement in surgical efficiency, the realization of safer surgery, and the like, for example.

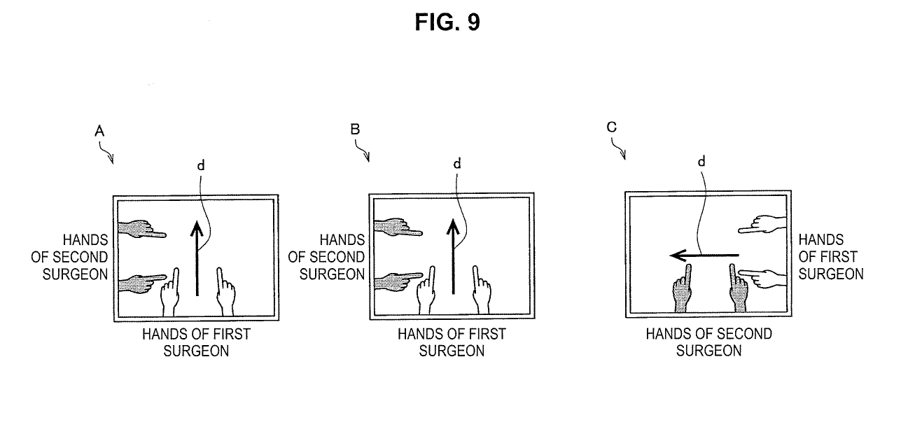

FIG. 9 is an explanatory diagram for explaining an overview of the image processing method according to the present embodiment. FIG. 9 illustrates one example of medical captured images displayed on the display screens of each of the display apparatus 200A and 200B in the case in which the display apparatus 200A and 200B are arranged as illustrated in FIG. 3. A of FIG. 9 illustrates one example of the medical captured image displayed on the display screen of the display apparatus 200A illustrated in FIG. 3. B of FIG. 9 illustrates one example of the medical captured image displayed on the display screen of the display apparatus 200B illustrated in FIG. 3 in the case in which the medical captured image is not rotated by the processes related to the image processing method according to the present embodiment. C of FIG. 9 illustrates one example of the medical captured image displayed on the display screen of the display apparatus 200B illustrated in FIG. 3 in the case in which the medical captured image is rotated by the processes related to the image processing method according to the present embodiment.

At the position of the first surgeon illustrated in FIG. 3, the top and bottom of the imaging device, the direction in which the first surgeon is facing, and the direction in which the display apparatus 200A is disposed are aligned. In this case, the medical observation apparatus 100 does not rotate the medical captured image corresponding to the display apparatus 200A. Therefore, as illustrated in A of FIG. 9, the first surgeon's own hands appear at the bottom of the display screen of the display apparatus 200A disposed in front of the first surgeon, and the first surgeon is able to carry out procedures without a sense of discomfort.

At the position of the second surgeon illustrated in FIG. 3, the top and bottom of the imaging device, the direction in which the second surgeon is facing, and the direction in which the display apparatus 200B is disposed are not aligned. Therefore, in the case of causing the display apparatus 200B to display the medical captured image as-is, as illustrated in B of FIG. 9, the second surgeon's own hands do not appear at the bottom of the display screen of the display apparatus 200B disposed in front of the second surgeon, and the vertical direction inside the display screen is misaligned with the direction in which the second surgeon is facing. For this reason, there is a possibility that the second surgeon may be unable to carry out procedures intuitively.

On the other hand, by having the medical observation apparatus 100 treat the display apparatus 200B as the target display apparatus and rotate the medical captured image corresponding to the display apparatus 200B, as illustrated in C of FIG. 9, the second surgeon's own hands appear at the bottom of the display screen of the display apparatus 200B disposed in front of the second surgeon, and in this case, the second surgeon is able to carry out procedures without a sense of discomfort.

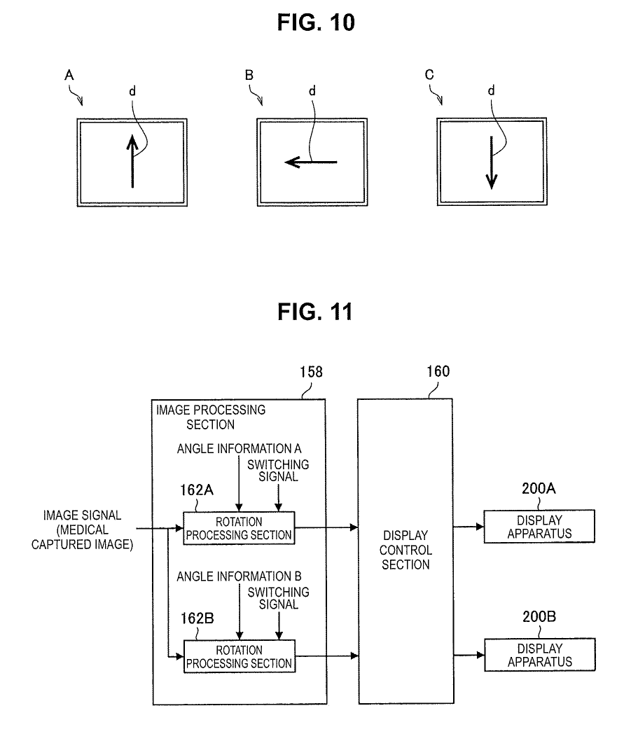

FIG. 10 is an explanatory diagram for explaining an overview of the image processing method according to the present embodiment. FIG. 10 illustrates one example of medical captured images displayed on the display screens of each of the display apparatus 200A, 200B, and 200C in the case in which the display apparatus 200A, 200B, and 200C are arranged as illustrated in FIG. 5. A of FIG. 10 illustrates one example of the medical captured image displayed on the display screen of the display apparatus 200A illustrated in FIG. 5. B of FIG. 10 illustrates one example of the medical captured image displayed on the display screen of the display apparatus 200B illustrated in FIG. 5 in the case in which the medical captured image is rotated by the processes related to the image processing method according to the present embodiment. C of FIG. 10 illustrates one example of the medical captured image displayed on the display screen of the display apparatus 200C illustrated in FIG. 5 in the case in which the medical captured image is rotated by the processes related to the image processing method according to the present embodiment. The sign d illustrated in FIG. 10 indicates the vertical direction of the imaging device, or in other words, the direction of the medical captured image captured by the imaging device.

As described above, the arrangement relationship of the display apparatus 200A and 200B illustrated in FIG. 5 and the arrangement relationship of the display apparatus 200A and 200B illustrated in FIG. 3 are the same. Therefore, as described with reference to A of FIG. 9 and C of FIG. 9, the medical observation apparatus 100 does not rotate the medical captured image corresponding to the display apparatus 200A, and rotates the medical captured image corresponding to the display apparatus 200B. Therefore, it is possible to match the vertical direction inside the display screen of each of the display apparatus 200A and 200B to the direction in which each of the surgeons looking at each of the display apparatus 200A and 200B is facing.

Also, as described above, in the example illustrated in FIG. 5, the angle obtained between the display apparatus 200A and the display apparatus 200C is set to 180[°]. Therefore, in the case of treating the display apparatus 200C as the target display apparatus, as illustrated in C of FIG. 10, the medical observation apparatus 100 rotates the medical captured image corresponding to the display apparatus 200C 180[°] with reference to the medical captured image illustrated in A of FIG. 10. Therefore, it is possible to match the vertical direction inside the display screen of the display apparatus 200C to the direction in which the medical personnel member looking at the display apparatus 200C is facing.

Note that in FIG. 10, as illustrated in B of FIG. 10 and C of FIG. 10, an example of rotating the medical captured image 90[°] counter-clockwise (or an example of rotating the medical captured image 270[°] clockwise; B of FIG. 10) and an example of rotating the medical captured image 180[°] counter-clockwise (or an example of rotating the medical captured image 180[°] clockwise; C of FIG. 10) are illustrated. However, the angle by which the medical observation apparatus 100 rotates the medical captured image is not limited to the examples illustrated in FIG. 10.

For example, the medical observation apparatus 100 is able to rotate the medical captured image counter-clockwise or clockwise according to the angle detected by the angle sensor according to the present embodiment. To give one example, in the case in which the angle detected by an absolute rotary encoder (one example of the angle sensor according to the present embodiment; the same applies hereinafter) is 40[°], the medical observation apparatus 100 rotates the medical captured image 40[°] counter-clockwise. Also, to give another example, in the case in which the angle detected by the absolute rotary encoder is 80[°], the medical observation apparatus 100 rotates the medical captured image 80[°] counter-clockwise. Note that although the above illustrates a case in which the angle sensor according to the present embodiment is an absolute rotary encoder as an example, as described above, the angle sensor according to the present embodiment may also be an incremental rotary encoder, an imaging device, or the like.

### [2-2] Processes Related to Image Processing Method According to Present Embodiment

Next, the processes related to the image processing method according to the present embodiment will be described more specifically.

The medical observation apparatus 100 processes the medical captured image in correspondence with the target display apparatus and rotates the medical captured image corresponding to the target display apparatus.

More specifically, the medical observation apparatus 100 rotates the medical captured image on the basis of the relative arrangement relationship of the target display apparatus with respect to a set primary display apparatus. In the case in which multiple target display apparatus exist, for each target display apparatus, the medical observation apparatus 100 rotates the medical captured image corresponding to the target display apparatus on the basis of the relative arrangement relationship with respect to the primary display apparatus.

The primary display apparatus according to the present embodiment is a display apparatus 200 that serves as a reference for the relative arrangement relationship of the display apparatus 200. The primary display apparatus may be a preset fixed display apparatus 200 or may be set in any way by an operation by a user who uses the medical observation system 1000. The examples described with reference to FIGS. 9 and 10 are examples in which the display apparatus 200A is set as the primary display apparatus.

As illustrated with reference to FIGS. 2 to 5, it is possible to specify the relative arrangement relationship of the display apparatus 200 according to the relative angle between the display apparatus 200. Therefore, by rotating the medical captured image corresponding to the target display apparatus in correspondence with the relative angle of the target display apparatus with respect to the primary display apparatus, the medical observation apparatus 100 realizes a rotation of the medical captured image based on the relative arrangement relationship of the target display apparatus with respect to the primary display apparatus.

As a specific example, a case in which the display apparatus 200A and 200B are arranged as illustrated in FIG. 3 and the display apparatus 200A is the primary display apparatus will be described.

For example, in the case in which the reference for the relative arrangement relationship of the display apparatus 200A and the display apparatus 200B is the first arrangement relationship above, the relative angle between the display apparatus 200A and 200B, namely the angle a, is 90[°] (treating the counter-clockwise direction as positive). In this case, when the display apparatus 200B is treated as the target display apparatus, the medical observation apparatus 100 rotates the medical captured image corresponding to the display apparatus 200B 180−a=90[°].

Also, for example, in the case in which the reference for the relative arrangement relationship of the display apparatus 200A and the display apparatus 200B is the second arrangement relationship above, the relative angle between the display apparatus 200A and 200B, namely the angle b, is −90[°] (treating the counter-clockwise direction as positive). In this case, when the display apparatus 200B is treated as the target display apparatus, the medical observation apparatus 100 rotates the medical captured image corresponding to the display apparatus 200B—b=90[°].

Therefore, on the display screen of the display apparatus 200B, the medical captured image rotated as illustrated in C of FIG. 9 for example is displayed. Note that, as described above, the medical observation apparatus 100 is able to rotate the medical captured image counter-clockwise or clockwise according to the angle detected by the angle sensor according to the present embodiment. In other words, the rotational angle by which the medical observation apparatus 100 rotates the medical captured image is not limited to the example described with reference to FIG. 3.

Note that the “process of rotating the medical captured image corresponding to the target display apparatus in correspondence with the relative angle of the target display apparatus with respect to the primary display apparatus” is not limited to the examples illustrated above.

For example, the medical observation apparatus 100 is also able to rotate the medical captured image discontinuously according to the angle detected by the angle sensor according to the present embodiment. To give one example of the case of rotating discontinuously when the reference for the relative arrangement relationship of the display apparatus 200A and the display apparatus 200B is the second arrangement relationship above, the medical observation apparatus 100 rotates the medical captured image as below according to the relative angle of the target display apparatus with respect to the primary display apparatus. Note that examples of the cause of rotating the medical captured image discontinuously obviously are not limited to the examples illustrated below.
Case in which the relative angle of the target display apparatus with respect to the primary display apparatus is from 271[°] to 45[°]: do not rotate the medical captured image
Case in which the relative angle of the target display apparatus with respect to the primary display apparatus is from 46[°] to 135[°]: rotate the medical captured image −90[°]
Case in which the relative angle of the target display apparatus with respect to the primary display apparatus is from 136[°] to 225[°]: rotate the medical captured image −180[°]
Case in which the relative angle of the target display apparatus with respect to the primary display apparatus is from 226[°] to 270[°]: rotate the medical captured image −270[°]

The above cites the case in which the reference for the relative arrangement relationship between the display apparatus 200A and the display apparatus 200B is the first arrangement relationship above or the second arrangement relationship above as examples, but the medical observation apparatus 100 rotates the medical captured image on the basis of the relative arrangement relationship of the display apparatus 200 regardless of how the reference is obtained or how the rotation is performed.

In the case in which the display apparatus 200 set as the primary display apparatus is set as the target display apparatus, the medical observation apparatus 100 does not rotate the medical captured image corresponding to the primary display apparatus.

For example, by not executing the rotation process based on the relative angle between the display apparatus 200 on the display apparatus 200 set as the primary display apparatus, the medical observation apparatus 100 does not rotate the medical captured image corresponding to the primary display apparatus.

Also, in the case in which the display apparatus 200 set as the primary display apparatus is set as the target display apparatus, the medical observation apparatus 100 may execute the rotation process based on the relative angle between the display apparatus 200. In this case, since the primary display apparatus and the target display apparatus are the same display apparatus 200, the relative angle between the primary display apparatus and the target display apparatus is 0[°]. Therefore, even in the case of executing the rotation process based on the relative angle between the display apparatus 200, the medical captured image corresponding to the primary display apparatus is not rotated.

Note that the medical observation apparatus 100 may also rotate the medical captured image corresponding to the primary display apparatus according to the positional relationship between the primary display apparatus and a medical personnel member associated with the primary display apparatus. The positional relationship between the primary display apparatus and the medical personnel member associated with the primary display apparatus is specified by any method capable of specifying the positional relationship, such as a method that uses a position sensor worn by the medical personnel member or a method that analyzes a captured image captured by an imaging device such as a surgical field camera, for example.

For example, in the “case in which the medical captured image corresponding to the primary display apparatus is rotated according to the positional relationship between the primary display apparatus and a medical personnel member associated with the primary display apparatus” as above, the medical observation apparatus 100 additionally rotates the medical captured image corresponding to an other target display apparatus by the amount of rotation of the medical captured image corresponding to the primary display apparatus.

Note that the processes related to the image processing method according to the present embodiment are not limited to the examples illustrated above.

For example, the medical observation apparatus 100 may activate or deactivate the function of processing the medical captured image in correspondence with the target display apparatus on the basis of a predetermined operation. In other words, it is also possible for the medical observation apparatus 100 to selectively switch whether or not to execute the process of rotating the medical captured image corresponding to the target display apparatus.

The predetermined operation according to the present embodiment may be, for example, an operation of switching the above function performed on an operating device provided in the medical observation apparatus 100, an operation of switching the above function performed on an external operating device such as a remote controller or a footswitch, or the like.

For example, in response to a switching signal corresponding an operation of switching the above function, the medical observation apparatus 100 activates the function and executes the process of rotating the medical captured image corresponding to the target display apparatus, or deactivates the function and does not execute the process of rotating the medical captured image corresponding to the target display apparatus. The switching signal according to the present embodiment may be, for example, a signal that expresses the activation or deactivation of the function according to the signal level (for example, the switching signal at the high level indicates activation of the function while the switching signal at the low level indicates deactivation of the function). Note that the switching signal according to the present embodiment obviously is not limited to the example illustrated above.

FIG. 11 is an explanatory diagram for explaining one example of the processes related to the image processing method according to the present embodiment, and uses the function blocks illustrated in FIG. 8 to illustrate one example of the processes related to the image processing method in the case in which the target display apparatus is the display apparatus 200A and 200B. In FIG. 11, the image processing section 158 and the display control section 160 illustrated in FIG. 8 are illustrated, while in addition, the display apparatus 200A and 200B are illustrated as one example of the target display apparatus.

The image processing section 158 illustrated in FIG. 11 includes a rotation processing section 162A corresponding to the display apparatus 200A and a rotation processing section 162B corresponding to the display apparatus 200B. In other words, the image processing section 158 illustrated in FIG. 11 includes a rotation processing section corresponding to each target display apparatus.

The rotation processing section 162A rotates the medical captured image indicated by the image signal on the basis of angle information A indicating the relative angle of the display apparatus 200A with respect to the primary display apparatus. For example, the rotation processing section 162A rotates the medical captured image by an amount equal to the angle indicated by the angle information A.

As described above, in the case in which the display apparatus 200A is the primary display apparatus, the rotation processing section 162A does not rotate the medical captured image. Also, as described above, in the case in which the medical captured image corresponding to the primary display apparatus is rotated, the rotation processing section 162A additionally rotates the medical captured image by the amount of rotation of the medical captured image corresponding to the primary display apparatus.

Also, in the case in which the switching signal is transmitted, for example, the rotation processing section 162A sets an operating mode that executes the process of rotating the medical captured image according to the switching signal or sets an operating mode that does not execute the process of rotating the medical captured image according to the switching signal. Subsequently, the process of rotating the medical captured image is executed selectively in accordance with the set operating mode.

The rotation processing section 162A transmits an image signal expressing the processed medical captured image to the display control section 160. Subsequently, the display control section 160 causes the medical captured image expressed by the image signal transmitted from the rotation processing section 162A to be displayed on the display screen of the display apparatus 200A.

Note that although FIG. 11 illustrates an example in which a single image signal is input into the rotation processing section 162A, an image signal expressing a medical captured image for the right eye and an image signal expressing a medical captured image for the left eye may also be input into the rotation processing section 162A. In this case, the rotation processing section 162A executes the process described with reference to FIG. 11 on each of the image signal expressing a medical captured image for the right eye and the image signal expressing a medical captured image for the left eye.

The rotation processing section 162B rotates the medical captured image indicated by the image signal on the basis of angle information B indicating the relative angle of the display apparatus 200B with respect to the primary display apparatus. For example, the rotation processing section 162B rotates the medical captured image by an amount equal to the angle indicated by the angle information B.

In the case in which the display apparatus 200B is the primary display apparatus, the rotation processing section 162B does not rotate the medical captured image, similarly to the rotation processing section 162A. Also, similarly to the rotation processing section 162A, in the case in which the medical captured image corresponding to the primary display apparatus is rotated, the rotation processing section 162B additionally rotates the medical captured image by the amount of rotation of the medical captured image corresponding to the primary display apparatus.

Also, in the case in which the switching signal is transmitted, the rotation processing section 162B selectively executes the process of rotating the medical captured image in accordance with the operating mode set according to the switching signal, similarly to the rotation processing section 162A.

The rotation processing section 162B transmits an image signal expressing the processed medical captured image to the display control section 160. Subsequently, the display control section 160 causes the medical captured image expressed by the image signal transmitted from the rotation processing section 162B to be displayed on the display screen of the display apparatus 200B.

Note that although FIG. 11 illustrates an example in which a single image signal is input into the rotation processing section 162B, an image signal expressing a medical captured image for the right eye and an image signal expressing a medical captured image for the left eye may also be input into the rotation processing section 162B. In this case, the rotation processing section 162B executes the process described with reference to FIG. 11 on each of the image signal expressing a medical captured image for the right eye and the image signal expressing a medical captured image for the left eye.

As illustrated in FIG. 11 for example, by including a rotation processing section corresponding to each target display apparatus, the image processing section 158 rotates the medical captured image on the basis of the relative arrangement relationship for each target display apparatus. Note that the functional configuration of the image processing section 158 obviously is not limited to the example illustrated in FIG. 11.

### [3] Example of Advantageous Effects Exhibited by Use of Image Processing Method According to Present Embodiment

By using the image processing method according to the present embodiment, the advantageous effects illustrated below are exhibited, for example. Note that the advantageous effects exhibited by using the image processing method according to the present embodiment obviously are not limited to the examples illustrated below.
By using the image processing method according to the present embodiment, it becomes possible to match the vertical direction inside the display screen to the direction in which a surgeon is facing, and therefore even with an arrangement of the display apparatus 200 as illustrated in FIG. 3, not only the first surgeon but also the second surgeon is able to look at the medical captured image in the correct direction.
Since the surgeons are able to look at the medical captured image in the correct direction, the surgeons are able to carry out procedures intuitively.
Hypothetically, even if the arrangement of the display apparatus 200 changes during surgery, since the medical captured image is rotated by the processes related to the image processing method according to the present embodiment, the surgeons are able to continue carrying out procedures intuitively. Also, in this case, since a special change of settings or the like is unnecessary, no time is needed to change settings or the like, potentially contributing to shortened surgery time.

### Program According to Present Embodiment

By having a program (for example, a program capable of executing the processes related to the image processing method according to the present embodiment) for causing a computer system to function as the medical observation apparatus according to the present embodiment (or the medical image processing apparatus according to the present embodiment) be executed by a processor or the like in the computer system, it is possible to potentially achieve an improvement in convenience for medical personnel members. At this point, the computer system according to the present embodiment may be a single computer or multiple computers. A series of processes related to the image processing method according to the present embodiment is executed by the computer system according to the present embodiment.

Additionally, by having the program for causing a computer system to function as the medical observation apparatus according to the present embodiment (or the medical image processing apparatus according to the present embodiment) be executed by a processor or the like in the computer system, the advantageous effects exhibited by the display realized by the processes related to the image processing method according to the present embodiment described above can be exhibited.

It should be understood by those skilled in the art that various modifications, combinations, sub-combinations and alterations may occur depending on design requirements and other factors insofar as they are within the scope of the appended claims or the equivalents thereof.

For example, although the above illustrates the provision of a program (computer program) for causing a computer system to function as the medical observation apparatus according to the present embodiment (or the medical image processing apparatus according to the present embodiment), in the present embodiment, the above program may also be provided in conjunction with a recording medium on which the above program is stored.

The configuration described above illustrates one example of the present embodiment, and rightfully belongs to the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1) A medical image processing apparatus including:

an image processing section including a function of processing a medical captured image captured by an imaging device that images an observation target in correspondence with a target display apparatus, in which

the image processing section rotates the medical captured image on the basis of a relative arrangement relationship of the target display apparatus with respect to a set primary display apparatus.

(2) The medical image processing apparatus according to (1), in which

in a case in which multiple target display apparatus exist, the image processing section rotates the medical captured image on the basis of the relative arrangement relationship for each of the target display apparatus.

(3) The medical image processing apparatus according to (1) or (2), in which

in a case in which the target display apparatus is the primary display apparatus, the image processing section does not rotate the medical captured image.

(4) The medical image processing apparatus according to any one of (1) to (3), in which

the image processing section rotates the medical captured image in correspondence with a relative angle of the target display apparatus with respect to the primary display apparatus.

(5) The medical image processing apparatus according to (4), in which

the relative angle is specified on the basis of a detection result of one or multiple angle sensors.

(6) The medical image processing apparatus according to any one of (1) to (5), in which

the image processing section activates or deactivates the function on the basis of a predetermined operation.

(7) The medical image processing apparatus according to any one of (1) to (6), further including:

a display control section configured to control a display of the medical captured image on a display screen of the target display apparatus.

(8) A medical observation apparatus including:

an imaging device configured to image an observation target; and

an image processing section including a function of processing a medical captured image captured by the imaging device in correspondence with a target display apparatus, in which

the image processing section rotates the medical captured image on the basis of a relative arrangement relationship of the target display apparatus with respect to a set primary display apparatus.

(9) The medical observation apparatus according to (8), further including:

an arm including multiple links joined to each other by one or multiple joint sections, in which

the imaging device is supported by the arm.

(10) The medical observation apparatus according to (8), in which

the imaging device is inserted into an inside of a body of a patient and images the inside of the body as the observation target.

(11) An image processing method, executed by a medical image processing apparatus, including:

processing a medical captured image captured by an imaging device that images an observation target in correspondence with a target display apparatus, in which

the processing rotates the medical captured image on the basis of a relative arrangement relationship of the target display apparatus with respect to a set primary display apparatus.

1-11. (canceled)

12: A medical image processing apparatus comprising:
processing circuitry; and
a memory storing instructions that when executed by the processing circuitry causes the processing circuitry to be configured to:
process a medical captured image captured by an imaging device that images an observation target in correspondence with a target display apparatus,
wherein the processing circuitry is further configured to process the medical captured image by being configured to rotate the medical captured image on a basis of a relative arrangement relationship of the target display apparatus with respect to a set primary display apparatus.

13: The medical image processing apparatus according to claim 12, wherein
in a case in which multiple target display apparatuses exist, the processing circuitry is further configured to process the medical captured image by being configured to rotate the medical captured image on the basis of the relative arrangement relationship for each of the target display apparatuses.

14: The medical image processing apparatus according to claim 12, wherein
in a case in which the target display apparatus is the primary display apparatus, the processing circuitry is further configured to process the medical captured image by not rotating the medical captured image.

15: The medical image processing apparatus according to claim 12, wherein
the processing circuitry is further configured to process the medical captured image by rotating the medical captured image in correspondence with a relative angle of the target display apparatus with respect to the primary display apparatus.

16: The medical image processing apparatus according to claim 15, wherein
the relative angle is specified on a basis of a detection result of one or multiple angle sensors.

17: The medical image processing apparatus according to claim 12, wherein
the processing circuitry is further configured to activate or deactivate the function on a basis of a predetermined operation.

18: The medical image processing apparatus according to claim 12, wherein
the processing circuitry is further configured to control a display of the medical captured image on a display screen of the target display apparatus.

19: A medical observation apparatus comprising:
an imaging device configured to image an observation target; and
a memory storing instructions that when executed by processing circuitry causes the processing circuitry to be configured to:
process a medical captured image captured by the imaging device in correspondence with a target display apparatus,
wherein the processing circuitry is further configured to process the medical captured image by being configured to rotate the medical captured image on a basis of a relative arrangement relationship of the target display apparatus with respect to a set primary display apparatus.

20: The medical observation apparatus according to claim 19, further comprising:
an arm including multiple links joined to each other by one or multiple joint sections, wherein
the imaging device is supported by the arm.

21: The medical observation apparatus according to claim 19, wherein
the imaging device is inserted into an inside of a body of a patient and images the inside of the body as the observation target.

22: An image processing method, executed by a medical image processing apparatus, comprising:
processing a medical captured image captured by an imaging device that images an observation target in correspondence with a target display apparatus, wherein
the processing rotates the medical captured image on a basis of a relative arrangement relationship of the target display apparatus with respect to a set primary display apparatus.
